(19) United States
(12) Patent Application Publication
(10) Pub. No.: US 2025/0205083 A1
(43) Pub. Date: Jun. 26, 2025

(54) ADJUSTABLE SHUNTING SYSTEMS WITH WAX ACTUATORS AND ASSOCIATED SYSTEMS, DEVICES, AND METHODS

(71) Applicant: Shifamed Holdings, LLC, Campbell, CA (US)

(72) Inventor: Tom Saul, Portland, OR (US)

(21) Appl. No.: 18/844,357

(22) PCT Filed: Mar. 17, 2023
(86) PCT No.: PCT/US2023/064636

§ 371 Date: Sep. 5, 2024

Related U.S. Application Data
(60) Provisional application No. 63/324,712, filed on Mar. 29, 2022.

(51) Int. Cl. A61F 9/007

(52) U.S. Cl. CPC A61F 9/00781

(57) ABSTRACT

The present technology is generally directed to adjustable shunting systems, including adjustable shunting systems having wax actuators. In some embodiments, an adjustable shunting system includes a wax actuator and a diaphragm operably coupled to the wax actuator. The wax actuator can be configured to selectively control the flow of fluid through the adjustable shunting system by alternating between (i) heating a first end of the wax actuator to move the wax actuator to a first position and open a flow path through the system by biasing the diaphragm in a first direction, and (ii) heating a second end of the wax actuator to move the wax actuator to a second position and close the flow path through the system by biasing the diaphragm in a second direction different than the first direction.

### CROSS-REFERENCE TO RELATED APPLICATION(S)

The present application claims priority to U.S. Provisional Patent Application No. 63/324,712, filed Mar. 29, 2022, the disclosure of which is incorporated by reference herein in its entirety.

### TECHNICAL FIELD

The present technology generally relates to implantable medical devices and, in particular, to adjustable shunting systems and associated methods for selectively controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma by draining aqueous from an anterior chamber of a patient's eye. The flow of fluid through these shunting systems is primarily controlled by the pressure gradient across the shunt and the physical characteristics of the flow path defined through the shunt (e.g., the resistance of the shunt lumen). Conventional, early shunting systems for treatment of glaucoma (sometimes referred to as minimally invasive glaucoma shunts or “MIGS”) have shown clinical benefit; however, there is a need for improved shunting systems and techniques for addressing elevated intraocular pressure and risks associated with glaucoma. For example, most conventional shunts cannot be adjusted after implantation and, therefore, cannot be adjusted or titrated to meet the patient's individual and variable needs and/or to account for changes in flow-related characteristics, such as flow volume, inflow pressure, and/or outflow resistance. As another example, there is a need for a shunting system able to be modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is a partially schematic illustration of an adjustable shunting system configured in accordance with embodiments of the present technology.

FIG. 1B is a perspective view of a flow control assembly of the adjustable shunting system of FIG. 1A.

FIGS. 1C and 1D are side views of the flow control assembly of FIG. 1B in a first configuration and a second configuration, respectively, in accordance with embodiments of the present technology.

FIGS. 2A and 2B are side views of another flow control assembly in a first configuration and a second configuration, respectively, and configured in accordance with embodiments of the present technology.

FIGS. 3A-3C are side views of an actuation assembly in a first configuration, an intermediate configuration, and a second configuration, respectively, and configured in accordance with embodiments of the present technology.

FIG. 4 is a perspective view of an actuation assembly manifold including a plurality of actuation assemblies and configured in accordance with embodiments of the present technology.

FIG. 5 is a perspective view of a plurality of actuation components configured in accordance with embodiments of the present technology.

### DETAILED DESCRIPTION

The present technology is generally directed to adjustable shunting systems, including adjustable shunting systems with wax actuators, and associated systems, devices, and methods. In at least some embodiments, a shunting system includes an actuation assembly having a wax actuator operably coupled to a diaphragm. The wax actuator can include a first end and a second end opposite the first end. The wax actuator can change volume when transitioned between material phases. For example, the wax actuator can increase in volume when transitioning from a solid phase to a liquid phase (e.g., melting), and can decrease in volume when transitioning from a liquid phase to a solid phase (e.g., cooling or solidifying). The wax actuator can be positioned within an interior of the actuation assembly, such that the changes in the volume of the wax actuator can redistribute the wax within the interior of the actuation assembly and cause corresponding changes to the configuration of the diaphragm (e.g., flexing, bending, deformation, and the like). Heating the first end of the wax actuator can create a temperature differential across the wax actuator and cause the second end to solidify before the first end, such that the second end can recruit material from the first end as the first end solidifies, which can draw material from the first end toward the second end and apply a corresponding force (e.g., a pressure) to the diaphragm to cause the diaphragm to flex toward the first (e.g., heated) end (e.g., in response to the displacement of the wax). Additionally, heating the second end of the wax actuator can cause the first end to solidify before the second end, such that the first end can recruit material from the second end as the second end solidifies, which can draw the second end toward the first end and apply a corresponding force (e.g., pressure) to the diaphragm and cause the diaphragm to flex away from the second end (e.g., in response to the displacement of the wax).

The wax actuator can selectively change a configuration of the diaphragm to control one or more flow characteristics (e.g., fluid resistance) through the shunting system. For example, the diaphragm can be transitioned between (i) a first configuration in which the diaphragm causes a flow path through the system to close and/or increases a fluid resistance of the flow path, and (ii) a second configuration in which the diaphragm causes the flow path to at least partially open and/or decreases a fluid resistance of the flow path. The first end of the wax actuator can be communicatively coupled to the diaphragm, such that the changes in pressure within the interior can transition the diaphragm between the first and second configurations. For example, heating the first end of the wax actuator can cause the diaphragm to bend toward the first end upon cooling (and re-solidification) of the wax actuator, thereby at least partially opening the flow path (e.g., the second configuration). Heating the second end of the wax actuator can cause the diaphragm to bend away from the second end upon cooling (and re-solidification) of the wax actuator, thereby closing the flow path (e.g., the first configuration). Accordingly, the wax actuator can be configured to selectively control the flow of fluid through the system by selectively alternating between (i) heating a first end of the wax actuator to open a flow path through the system, and (ii) heating a second end of the wax actuator to close the flow path through the system.

As described in greater detail below; it is expected that in at least some embodiments the present technology may exhibit one or more advantageous characteristics that improve operation of adjustable shunting systems. For example, wax actuators are expected to have a reduced likelihood of failure and/or are expected to operate reliably in a variety of conditions, including when positioned within a patient's eye. Additionally, adjustable shunting systems including wax actuators can be relatively easy and/or inexpensive to manufacture.

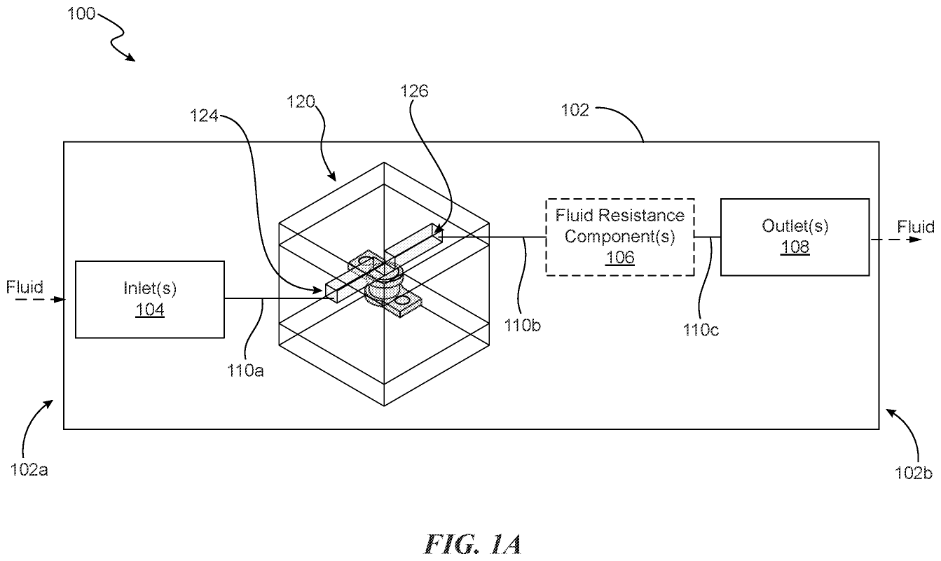

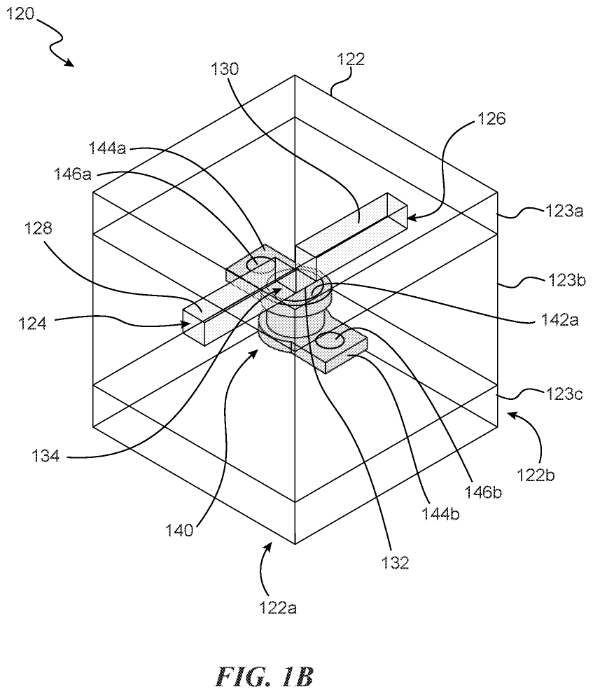

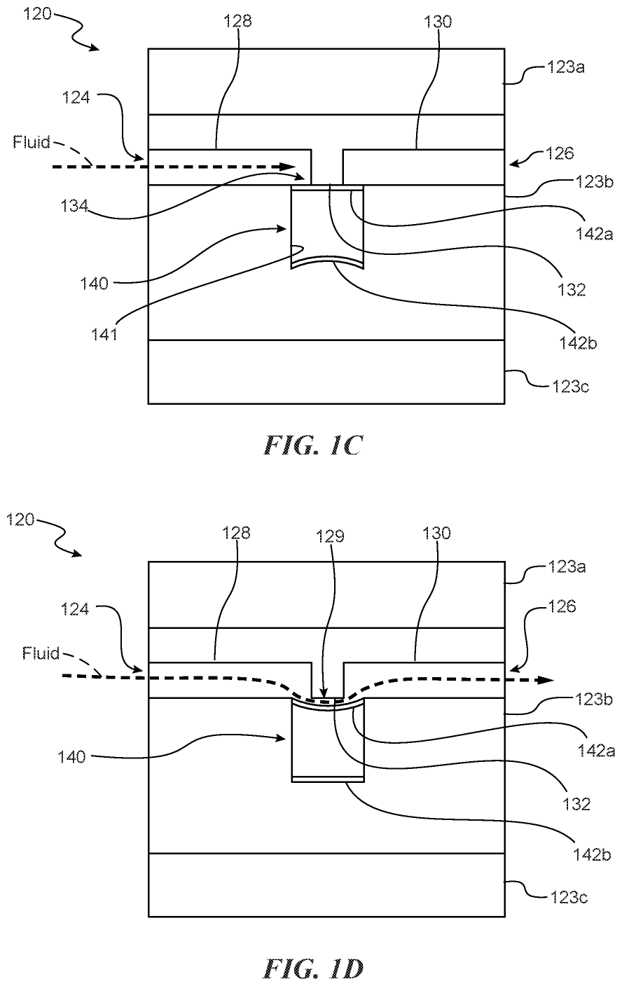

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-5.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art. Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIG. 1A is a partially schematic illustration of an adjustable shunting system 100 (“the system 100”) configured in accordance with embodiments of the present technology. As described in greater detail below, the system 100 is configured to provide an adjustable therapy for draining fluid from a first body region to a second body region, such as to drain aqueous from an anterior chamber of a patient's eye. The system 100 includes a housing 102, one or more first openings or inlets 104 (shown schematically), a flow control assembly 120, one or more second openings or outlets 108 (shown schematically), and one or more fluid conduits or channels 110a-c that extend between and fluidly couple individual elements (e.g., the inlets 104, the outlets 108, the flow control assembly 120, etc.) of the system 100.

The housing 102 (which can also be referred to as a casing, membrane, shunting element, or the like) includes a first end portion 102a and a second end portion 102b opposite the first end portion 102a. The housing 102 can be composed of a slightly elastic or flexible biocompatible material (e.g., silicone, etc.), or any other suitable material. The inlets 104 (e.g., individual ones of the inlets 104) can be positioned at or near the first end portion 102a of the housing 102. The outlets 108 (e.g., individual ones of the outlets 108) can be positioned at or near the second end portion 102b of the housing 102. The flow control assembly 120 (which can also be referred to as a flow control plate or a flow control cartridge) is positioned within the housing 102 and is configured to selectively control the flow of fluid through the system 100, such as between the inlet(s) 104 and the outlet(s) 108. In at least some embodiments, for example, an outer surface of the flow control assembly 120 forms a substantial fluid seal with an inner surface of the housing 102, such that fluid flowing into the system 100 generally must pass through the flow control assembly 120 before draining from the system. The operation of the flow control assembly 120 is described below regarding FIGS. 1B-1D.

The flow control assembly 120 can include a first fluid aperture or inlet 124 and a second fluid aperture or outlet 126. In the illustrated embodiment, the flow control assembly inlet 124 is fluidly coupled to one or more of the inlets 104 via a first fluid channel or conduit 110a, and the flow control assembly outlet 126 is fluidly coupled to one or more of the outlets 108 via a second fluid channel or conduit 110b. Accordingly, fluid that enters the system 100 via the inlet(s) 104 can flow toward the flow control assembly inlet 124, and fluid that exits the flow control assembly 120 via the flow control assembly outlet 126 can flow toward the outlet(s) 108 and drain from the system 100.

In some embodiments, the system 100 can include one or more fluid resistance components 106 (shown schematically). In the illustrated embodiment, the fluid resistance components 106 are positioned between the flow control assembly 120 and the outlet(s) 108. In other embodiments, one or more of the fluid resistance components 106 can be positioned between the inlet(s) 104 and the flow control assembly 120, and/or have any other suitable position. Individual ones of the fluid resistance components 106 can include, for example, a channel having at least one dimension (e.g., a length, a width, a circumference, a cross-sectional area, or the like) that corresponds to a fluid resistance of the channel, or any other suitable fluid resistance component. In some embodiments, for example, the fluid resistance components 106 includes one or more of the channels 110a-c fluidly coupling the inlets 104 to the flow control assembly inlet 124 and/or fluidly coupling the flow control assembly outlet 126 to the outlet(s) 108. Accordingly, the fluid resistance components 106 can form part of the fluid path through the system 100. Regardless of the structure of the fluid resistance components, in some embodiments the fluid resistance components 106 can provide all or substantially all of the fluid resistance of the adjustable shunting system 100. In other embodiments, a portion of the flow control assembly 120 may provide some fluid resistance in addition to the resistance provided by the fluid resistance components 106.

In operation, the system 100 can be positioned with the inlet(s) 104 in the first body region and the outlet(s) 108 in the second body region. The flow control assembly 120 can be actuated to allow fluid to flow from the first body region to the second body region. In other embodiments, the system can be positioned to operate in reverse, e.g., to allow fluid to enter via the outlet(s) 108 and exit via the inlet(s) 104. In some embodiments the system 100 can include more than one flow control assembly 120, such as at least two, three, or any other suitable number. In such embodiments, each of the flow control assemblies 120 can be fluidly coupled to one or more respective fluid resistance components 106 and individual ones of the respective fluid resistance components 106 can have a different fluid resistance; accordingly, each of the flow control assemblies 120 can be selectively and/or independently actuated to provide an adjustable, titratable therapy to a patient.

FIG. 1B is a perspective view of the flow control assembly 120 of the system 100 of FIG. 1A. The flow control assembly 120 can include a body 122 having a first end portion 122a and a second end portion 122b opposite the first end portion 122a. In the illustrated embodiment, the body 122 has a square cross-sectional shape. In other embodiments, however, the body 122 can have a rectilinear, circular, curvilinear, or any other suitable cross-sectional shape. The first end portion 122a can include the fluid inlet 124 (described previously with respect to FIG. 1A), and the second end portion 122b can include the fluid outlet 126 (described previously with respect to FIG. 1A). The fluid inlet 124 can be fluidly coupled to a first (e.g., inlet) channel or channel portion 128 (“the inlet channel 128”) that extends at least partially inwardly, e.g., away from the first end portion 122a and/or toward the second end portion 122b. The fluid outlet 126 can be fluidly coupled to a second (e.g., outlet) channel or channel portion 130 (“the outlet channel 130”) that extends at least partially inwardly, e.g., away from the second end portion 122b and/or toward the first end portion 122a. A valving or sealing surface 132 is positioned at least partially between the inlet channel 128 and the outlet channel 130. In some embodiments, the sealing surface 132 can be configured to selectively form a substantially fluid-impermeable seal 134, e.g., to at least partially or fully prevent fluid in the inlet channel 128 from flowing toward and/or entering the outlet channel 130, and/or to at least partially or fully prevent fluid in the outlet channel 130 from flowing toward and/or entering the inlet channel 128. In the illustrated embodiment, the fluid inlet 124, the fluid outlet 126, the inlet channel 128, and the outlet channel 130 each have a square shape. In other embodiments, each of the fluid inlet 124, the fluid outlet 126, the inlet channel 128, and the outlet channel 130 can have a rectangular, rectilinear, circular, curvilinear, or any other suitable shape.

The flow control assembly 120 can further include an actuation assembly 140. The actuation assembly 140 can be positioned at least partially between the inlet channel 128 and the outlet channel 130 and/or at least partially aligned with the sealing surface 132. The actuation assembly 140 can include one or more membranes or diaphragms. For example, in the illustrated embodiment and as best shown in FIGS. 1C and 1D, the actuation assembly 140 includes a first or upper diaphragm 142a and a second or lower diaphragm 142b opposite the first diaphragm 142a. In other embodiments, the actuation assembly 140 can include more or fewer diaphragms, such as one or more than two diaphragms, or any other suitable number of diaphragms. Each of the diaphragms 142a-b can be formed from a semi-flexible and substantially fluid-impermeable material. For example, the diaphragms 142a-b can be composed of a polymer, such as polydimethylsiloxane (PDMS), or any other suitable material or combination of suitable materials. Each of the diaphragms 142a-b can be configured to flex or bend relative to at least the sealing surface 132. As described in detail below, the diaphragms 142a-b are configured to flex or bend relative to the sealing surface 132 to alter fluid flow through the flow control assembly 120.

The actuation assembly 140 can further include one or more actuation components. As best shown in FIG. 1B, the actuation assembly 140 includes a first actuation component 144a and a second actuation component 144b. In other embodiments, the actuation assembly 140 can include more or fewer actuation components, such as a same number of actuation components as the number of diaphragms. Each of the actuation components 144a-b can be energetically (e.g., thermally) insulated/isolated from each other and operably coupled to one or more of the diaphragms 142a-b (also shown in FIGS. 1C and 1D), such that actuating the actuation components 144a-b can selectively and/or independently flex or bend one or more of the diaphragms 142a-b.

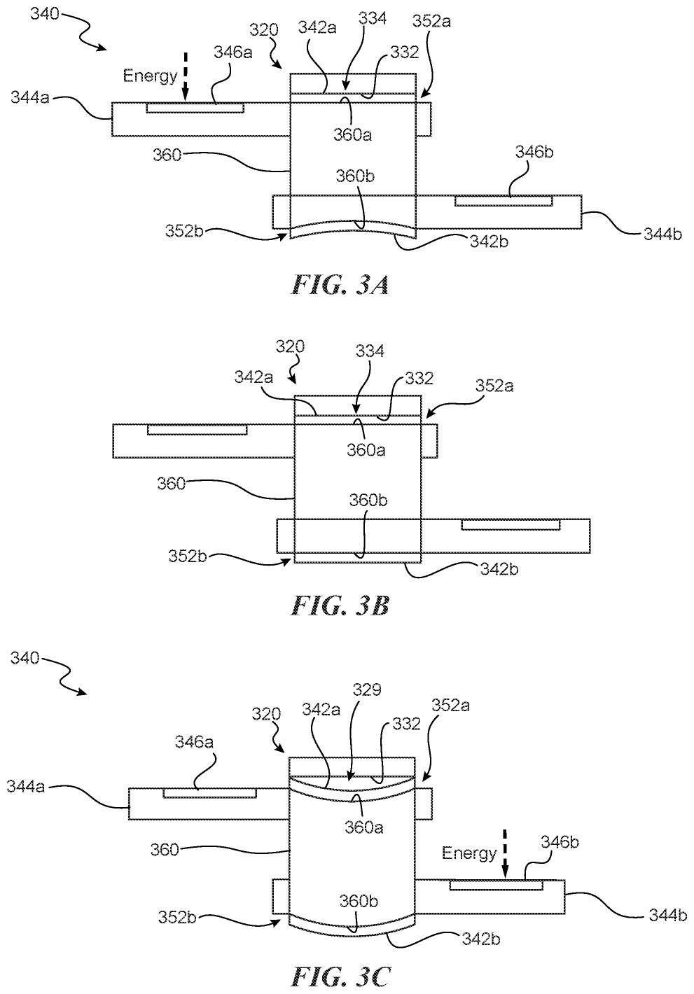

To actuate the actuation assembly 140 and bend/flex the diaphragms 142a-b, energy (e.g., light energy, heat energy, etc.) can be applied to individual ones of the actuation components 144a-b, as described below with respect to FIGS. 3A-3C. The energy can be applied non-invasively from a source external to the system 100 (FIG. 1A) and/or the patient. In some embodiments, one or more of the actuation components 144a-b includes a respective target or target region 146a-b for receiving the externally applied energy. In the illustrated embodiment, for example, the first actuation component 144a includes a first target 146a and the second actuation component 144b includes a second target 146b. The targets 146a-b can formed from a conductive material, such as metal, and may be treated to increase/enhance energy absorption, for example, by roughening the target's surfaces or applying an oxide layer over or all or part of the targets 146a-b. The actuation components 144a-b and/or the targets 146a-b can have a higher thermal conductivity than the body 122, for example, to improve the transmission of the applied energy from the actuation components 144a-b and/or the targets 146a-b to the actuation assembly 140. It is expected that actuation components that include targets will be easier to actuate via externally applied energy.

FIGS. 1C and 1D are side views of the flow control assembly 120 of FIG. 1B in a first configuration and a second configuration, respectively, in accordance with embodiments of the present technology. In particular, the flow control assembly 120 can transition between (i) a first configuration shown in FIG. 1C in which the actuation assembly 140 at least partially or fully prevents fluid from flowing between the inlet channel 128 and the outlet channel 130, and (ii) a second configuration shown in FIG. 1D in which the actuation assembly 140 allows fluid to flow between the inlet channel 128 and the outlet channel 130. Referring to FIG. 1C, in the first configuration, the first diaphragm 142a can at least partially contact or abut the sealing surface 132, e.g., to form the seal 134 and at least partially or fully prevent fluid from flowing between the inlet channel 128 and the outlet channel 130. Referring to FIG. 1D, in the second configuration, the first diaphragm 142a is flexed or bent relative to its position in the first configuration such that it is spaced apart from the sealing surface 132 (by, e.g., a gap 129). As indicated by the arrow, fluid can flow through the gap between the inlet channel 128 and the outlet channel 130 when the flow control assembly 120 is in the second configuration.

The first and second diaphragms 142a, 142b can flex in concert when the flow control assembly 120 transitions between the first and second configurations. In the illustrated embodiment, for example, the first and second diaphragms 142a, 142b both flex away from the sealing surface 132 (e.g., downwardly) when the flow control assembly 120 transitions from the first configuration (FIG. 1C) toward the second configuration (FIG. 1D). Similarly, the first and second diaphragms 142a, 142b can both flex toward the sealing surface 132 (e.g., upwardly) when the flow control assembly 120 transitions from the second configuration to the first configuration. In some aspects of the present technology, having diaphragms positioned on opposite sides of the actuation assembly 140 that flex in concert can improve the repeatability and/or the consistency with which the actuation assembly 140 can be actuated. As described in detail with respect to FIGS. 3A-a3C. the actuation assembly 140 can include a wax actuator that can be selectively actuated to flex the first and second diaphragms 142a, 142b, and to transition the flow control assembly 120 between the first and second configurations. The actuation assembly 140 can be positioned within a recess or chamber 141 formed in the flow control assembly 120. In the illustrated embodiment, for example, the chamber 141 is formed in the second plate 123b of the flow control assembly 120.

In some embodiments, the body 122 can include one or more plates or layers 123. In the illustrated embodiment, for example, the body 122 includes a first or upper plate 123a, a second or intermediate plate 123b, and a third or lower plate 123c. The first plate 123a and the third plate 123c can be positioned on opposite sides of the second plate 123b, e.g., to protect the second plate 123b from damage. The first plate 123a and the third plate 123c can be formed from a polymer, such as polymethylmethacrylate (PMMA), or any other suitable material or combination of suitable materials. In some embodiments, one or both of the first plate 123a and the third plate 123c can be omitted. The intermediate plate 123b can include some or all of the elements of the flow control assembly 120, such as the fluid inlet 124, the fluid outlet 126, the inlet channel 128, the outlet channel 130, the sealing surface 132, the actuation assembly 140, and/or any other suitable elements of the flow control assembly 120. The intermediate plate 123b can be formed from an elastomeric or otherwise flexible/deformable material such that the intermediate plate 123b can allow (or at least not substantially inhibit) the flexing of the diaphragms 142a, 142b. Additionally, or alternatively, one or more of the plates 123a-c can have a generally or substantially lower thermal conductivity than the targets 146a, 146b (FIG. 1B).

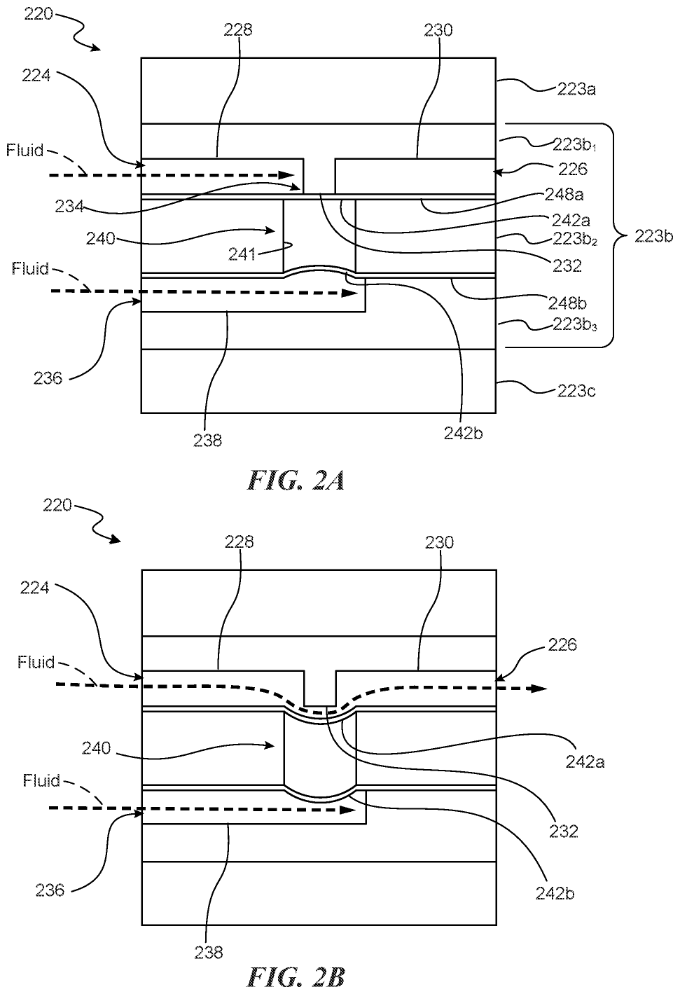

FIGS. 2A and 2B are side views of another flow control assembly 220 in a first configuration and a second configuration, respectively, and configured in accordance with embodiments of the present technology. Referring to FIGS. 2A and 2B together, the flow control assembly 220 can be generally similar to the flow control assembly 120 of FIGS. 1A-1D, with like numbers (e.g., sealing surface 232 versus the sealing surface 132 of FIGS. 1B-1D) indicating like elements. Relative to the flow control assembly 120, the flow control assembly 220 can further include a port 236 and an associated channel 238. The port 236 can receive fluid from one or more inlets of an adjustable shunting system, such as the inlets 104 of FIG. 1A. The channel 238 can be at least partially aligned with and/or communicatively coupled to the second diaphragm 242b. In at least some embodiments, for example, at least a portion of the second diaphragm 242b bends or deflect into the channel 238 when the flow control assembly 220 is in the second configuration (FIG. 2B). In the illustrated embodiment, the port 236 is not fluidly coupled to a corresponding fluid outlet, such that fluid that enters the flow control assembly 220 via the port 236 must also exit the flow control assembly 220 via the port 236 (e.g., the port 236 and the channel 238 are an inlet-outlet port and an inlet-outlet channel, respectively). In other embodiments, the port 236 can be fluidly coupled to a corresponding fluid outlet which can be generally similar to or the same as the outlet 226, such that the port 236 and the inlet 224 can define two distinct flow paths through the flow control assembly 220.

Fluid that enters the channel 238 via the port 236 can contact the second diaphragm 242b, e.g., to at least partially oppose (e.g., balance, counteract, equalized, mitigate, relieve or the like) one or more forces/pressures applied to the first diaphragm 242a by fluid that enters the channel 228 via the port 224. For example, a same body of fluid can interact with the first diaphragm 242a and the second diaphragm 242b (via, e.g., the inlets 104 of FIG. 1A), such that the fluid can exert a same or similar force/pressure on the first diaphragm 242a and the second diaphragm 242b. Accordingly, in at least some embodiments, the ports 224, 236 and the associated channels 228, 238 can cause opposing forces to be applied to the first and second diaphragms 242a, 242b, respectively, e.g., during operation of the flow control assembly 220. The fluid within the channel 238 can be more compliant than the material of the second plate 223b such that there can be reduced resistance in transitioning the actuation assembly 240 between the first configuration (FIG. 2A) and the second configuration (FIG. 2B). Additionally or alternatively, because the channel 238 allows the second diaphragm 242b to flex or bend, the second plate 223b and/or one or more of the layers 223b₁₋₃ thereof can be formed from a generally rigid, non-compliant, and/or non-elastomeric material. In some aspects of the present technology, the opposing forces and/or reduced resistance to transitioning can improve the repeatability and/or consistency with which the flow control assembly 220 can be transitioned between the first and second configurations.

Referring to FIG. 2A, one or more of the diaphragms 242a-b can be formed from a flexible or a corresponding deformable region or layer 248a-b positioned between and/or within individual ones of the plates 223a-c. In the illustrated embodiment, for example, the first diaphragm 242a is formed from a first flexible layer 248a positioned on a first side of (e.g., above) the actuation assembly 240 and the second diaphragm 242b is formed from a second flexible layer 248b positioned on a second or opposite side of (e.g., below) the actuation assembly 240. The first flexible layer 248a and the second flexible layer 248b can be positioned within the intermediate plate 223b and separate the intermediate plate 223b into a first layer or region 223b₁ above the first flexible layer 248a, a second layer or region 223b₂ between the first and second flexible layer 248a-b, and a third layer or region 223b₃ below the second flexible layer 248b. The first layer 223b₁ can include the sealing surface 232 and at least portions of the fluid inlet 224, the fluid outlet 226, the inlet channel 228, and the outlet channel 230. The first flexible layer 248a can be coupled to the first layer 223b₁ to form any remaining portions of the fluid inlet 224, the fluid outlet 226, the inlet channel 228, and/or the outlet channel 230. The second layer 223b₂ can include the actuation assembly 140. The third layer 223b₃ can include at least a portion of the port 236 and/or the channel 238; the second flexible layer 248b can be coupled to the third layer 223b₃ to form any remaining portions of the port 236 and the channel 238. In other embodiments, the first flexible layer 248a and/or the second flexible layer 248b can be contained at least partially or fully within the intermediate plate 223b.

FIGS. 3A-3C are side views of an actuation assembly 340 in a first configuration, an intermediate configuration, and a second configuration, respectively, and configured in accordance with embodiments of the present technology. The actuation assembly 340 can be generally similar to or the same as the actuation assembly 140 of FIGS. 1A-1D and/or the actuation assembly 240 of FIGS. 2A and 2B, with like numbers (actuation components 334a-b versus the actuation components 134a-b of FIG. 1B) indicating like elements. In FIGS. 3A-3C, the side views of the actuation assembly 340 have each been rotated 90 degrees relative to the side views of the actuation assembly 140 in FIGS. 1B and 1C and the actuation assembly 240 in FIGS. 2A and 2B. Additionally, in FIGS. 3A-3C, diaphragms 342a-b and sealing surface 332 are shown for purposes of clarity. As described previously, the actuation assembly 340 can be transitioned between (i) the first configuration (FIG. 3A) in which the first diaphragm 342a forms the seal 334 with the sealing surface 332 and (ii) the second configuration (FIG. 3C) in which the actuation assembly 340 allows fluid to flow through the gap 329 created between the first diaphragm 342a and the sealing surface 332. The intermediate configuration (FIG. 3B) illustrates one possible configuration of the actuation assembly 340 as it transitions between the first configuration (FIG. 3A) and the second configuration (FIG. 3C), e.g., to illustrate the operation of the actuation assembly 340. It will be appreciated that, in practice, other intermediate configurations may be possible and the actuation assembly 340 may not assume the intermediate configuration shown in FIG. 3B.

Referring collectively to FIGS. 3A-3C, the actuation assembly 340 can include a first (e.g., upper) end portion 352a and a second (e.g., lower) end portion 352b. A first actuation component 344a can be positioned proximate the first end portion 352a and a second actuation component 344b can be positioned proximate the second end portion 352b, opposite the first actuation component 344a. In at least some embodiments, the first end portion 352a includes at least part of the first diaphragm 342a and/or the second end portion 352b includes at least part of the second diaphragm 342b. The actuation assembly 340 can further include a wax actuator 360. In at least some embodiments, the wax actuator 360 can be positioned within an opening or chamber formed in the second plate of a flow control assembly, such as the chamber 141 formed in the second plate 123b (labeled in FIG. 1C) and/or the chamber 241 formed in the second plate 223b (e.g., the second layer 223b₁ of the second plate 223b; labeled in FIG. 2A). In other embodiments, the wax actuator 360 can be contained within a housing or casing positioned within a flow control assembly. In other embodiments, however, the wax actuator 360 can have any other suitable positions and/or containers. The wax actuator 360 can be composed of one or more biocompatible waxes, such as one or more paraffin waxes, and/or any other suitable wax and/or combinations thereof. The wax actuator 360 can be transitionable between a first material phase or state (e.g., a solid phase) and a second material phase or state (e.g., a liquid phase). The wax actuator 360 can be transitioned from the first material phase to the second material phase by applying energy (e.g., light energy, electrical energy, etc.) to the wax actuator 360 to heat the wax actuator 360 above a solid-liquid transition temperature. In some embodiments, the solid-liquid transition temperature can be within a temperature range of between about 38-42° C. to about 37-50° C. As the wax actuator 360 is heated above the solid-liquid transition temperature, the wax actuator 360 (or a portion thereof) can transition from the first (e.g., solid) material phase to the second (e.g., liquid) material phase. As the wax actuator 360 cools to below the solid-liquid transition temperature, the wax actuator 360 can transition from the second material phase to the first material phase. The wax actuator 360 can cool over time, e.g., in the absence of applied energy. Accordingly, after the energy is applied to transition the wax actuator 360 from the first (e.g., solid) material phase (e.g., FIGS. 3A and 3C) to the second (e.g., liquid) material phase (e.g., FIG. 3B), the wax actuator 360 can return to the first material phase as the wax actuator 360 cools over time.

The first actuation component 344a and the second actuation component 344b can be composed at least partially of an energy-conducting material or alloy (e.g., palladium, gold, etc.). The first actuation component 334a and the second actuation component 334b can be thermally coupled to the wax actuator 360, such that energy can be applied to the first actuation component 334a (e.g., the first target 346a) or the second actuation component 334b (e.g., the second target 346b) to transition the wax actuator 360 between material states, e.g., to heat the wax actuator 360 above its solid-liquid transition temperature. In the illustrated embodiment, for example, the first actuation component 334a is thermally coupled to at least a first end 360a of the wax actuator 360 and the second actuation component 334b is thermally coupled to at least a second end 360b of the wax actuator 360 opposite the first end 360a. Accordingly, energy can be applied to the first actuation component 334a to heat at least the first end 360a of the wax 360 and energy can be applied to the second actuation component 334b to heat at least the second end 360b of the wax 360.

The energy applied to one end (e.g., the first end 360a) of the wax actuator 360 can diffuse or spread toward an opposite end (e.g., the second end 360b) of the wax actuator 360, such that applying energy to first actuation component 334a or the second actuation component 334b can melt the entire wax actuator 360. The first end 360a and the second end 360b can have different melting rates depending on which actuation components 334a-b was heated. For example, if energy was applied to the first actuation component 334a, the first end 360a of the wax actuator 360 is expected to melt (e.g., reach the transition temperature) before the second end 360b. Likewise, if energy was applied to the second actuation component 334b, the second end 360b of the wax actuator 360 is expected to melt (e.g., reach the transition temperature) before the first end 360a. Depending on how long the energy is applied, the wax actuator 360 may nevertheless melt throughout its entirety regardless of which actuation component 334a-b is utilized due to heat spreading through the wax actuator 360 (e.g., substantially the entire wax actuator 360 transforms to a liquid phase). Once energy sufficient to melt the wax actuator 360 has been applied to the first end 360a or the second end 360b, the energy source can be deactivated and the wax actuator 360 can be allowed to cool. In some embodiments, the energy applied to the ends 360a, 360b can be generally or substantially constant over time. In other embodiments, the energy applied to the ends 360a, 360b can increase and/or decrease over time. The first end 360a and the second end 360b may have different cooling rates. For example, if energy was applied to the first actuation component 334a, the first end 360a of the wax actuator 360 is expected to cool slower than the second end 360b, such that the second end 360b of the wax actuator 360 can return to the solid phase before the first end 360a. As another example, if energy was applied to the second actuation component 334b, the second end 360b of the wax actuator 360 is expected to cool slower than the first end of the wax actuator 360 such that the first end 360a of the wax actuator 360 can return to the solid phase before the second end 360b. Additionally, or alternatively, the heated end of the wax is expected to have a temperature greater than the non-heated end, and thus solidify (e.g., cool below the transition temperature) after the other end of the wax due, at least partially, to the temperature differential created in the wax actuator by applying heat to the heated end. As described in detail below, this can cause the wax to migrate or be displaced and drive changes in the configuration of the diaphragms 342a-b.

The wax actuator 360 will undergo a volumetric change when the wax actuator 360 transitions between material phases. Generally, the wax actuator 360 can have a first volume in a solid phase and a second volume greater than the first volume in a liquid phase. Accordingly, the wax actuator 360 can transition from the first volume to the second volume when the wax actuator 360 melts and enters a liquid phase, and the wax can return from the second volume to the first volume when the wax returns to the solid phase. In some embodiments, the second volume can be between about 5% to about 15% greater than the first volume, or between about 7% to about 10% greater than the first volume. In other embodiments, the difference between the first and second volumes can be any other suitable difference configured to bend/flex the first diaphragm 342a such that the first diaphragm 342a can open and/or close the gap 329 (FIG. 3C). The wax actuator 360 can be contained within a finite volume (e.g., the chamber 141 of FIG. 1C), such that, when the wax actuator 360 transitions between phases, the associated increase or decrease in volume can produce a corresponding increase or decrease in pressure within the interior 354. Accordingly, increasing the volume of the wax actuator 360 (by, e.g., melting the wax actuator 360) can increase the pressure within the interior 354, and decreasing the volume of the wax actuator 360 (by, e.g., allowing the wax actuator 360 to cool) can decrease the pressure within the interior 354.

The wax actuator 360 can be operably coupled to the first diaphragm 342a and/or the second diaphragm 342b such that the pressure changes induced by the wax actuator 360 can flex and/or bend the diaphragms 342. In the illustrated embodiment, for example, the first end 360a of the wax actuator 360 contacts the first diaphragm 342a and the second end 360b of the wax actuator contacts the second diaphragms 342a-b. As the wax actuator 360 transitions between material phases, the associated displacement of the wax actuator 360 can apply a force to one or both of the diaphragms 342a, 342b and cause one or both of the diaphragms 342a, 342b to bend/flex, e.g., to transition the actuation assembly 340 between the first configuration (FIG. 3A) and the second configuration (FIG. 3C). For example, when the wax 360 transitions from the liquid phase (e.g., FIG. 3B) to the solid phase (e.g., FIG. 3C), the downward displacement of the wax 360 can produce a corresponding downward deflection in the first diaphragm 342a and/or the second diaphragm 342b. The volume of the wax actuator 360 in the liquid phase (FIG. 3B) can be generally similar to or the same as a volume of the chamber containing the wax actuator 360 (e.g., the chamber 141 of FIG. 1C), such that the wax actuator 360 occupies all or substantially all of the chamber in the liquid phase.

The actuation assembly 340 can be transitioned from the first configuration (FIG. 3A) to and/or toward the second configuration (FIG. 3C) by heating and cooling the wax actuator 360. Referring to FIG. 3A, in the illustrated embodiment, the actuation assembly 340 is in the first configuration and the wax actuator 360 is in a first material phase (e.g., solid). Energy sufficient to heat the wax actuator 360 above its solid-liquid transition temperature is applied to the first actuation component 344a, causing the wax actuator 360 to transition (e.g., melt) from the first material phase (FIG. 3A) to the second material phase (FIG. 3B). Referring to FIG. 3B, in the second material phase, the volume of the wax actuator 360 can increase, as described above. Because energy was applied proximate the first end 360a of the wax actuator 360, the second end 360b is expected to cool to/below the transition temperature and return to the first material phase before the first end 360a. Referring to FIG. 3C, as the second end 360b cools, it produces a corresponding decrease in the volume that can draw or recruit material from the first end 360a of the wax actuator 360 toward the second end 352b of the actuation assembly 340, e.g., causing the first end 360a to bend or “sink” downwardly as shown in FIG. 3C. This bending/sinking of the first end 360a can produce a corresponding bend in the first diaphragm 342a and create the gap 329 between the first diaphragm 342a the sealing surface 332. For example, the formation of “sink” in the first end 360a as the wax actuator cools may apply a force directly to the first diaphragm 342a and/or reduce the pressure within the interior 354 between the first end 360a and the first diaphragm 342a, such that the first diaphragm 342a is drawn inwardly toward the wax actuator 360. In some embodiments, as the second end 360b of the wax cools, the weight of the wax 360 and/or the downward migration of the wax 360 can bend or “sink” the second diaphragm 342b, as shown in FIG. 3C.

The actuation assembly 340 can be transitioned from the second configuration (FIG. 3C) to and/or toward the first configuration (FIG. 3A) by heating the wax actuator 360 in reverse. For example, referring to FIG. 3C in the illustrated embodiment, the actuation assembly 340 is in the second configuration and the wax actuator 360 is in a first material phase (e.g., solid). Energy sufficient to heat the wax actuator 360 above its solid-liquid transition temperature can be applied to the second actuation component 344b, causing the wax actuator 360 to transition (e.g., melt) from the first material phase (FIG. 3C) to the second material phase (FIG. 3B). Referring to FIG. 3B, in the second material phase, the volume of the wax actuator 360 can increase, as described above. This increase in volume can deflect the first diaphragm 342a upwardly and/or toward the sealing surface 332, e.g., to at least partially close the gap 329 (FIG. 3C) and/or at least partially form the seal 334. Because the energy is applied proximate the second end 360b of the wax actuator 360, the first end 360a is expected to cool to/below the transition temperature and return to the first material phase before the second end 360b. Referring to FIG. 3A, as the first end 360a cools, in can continue to deflect or force the first diaphragm 342a toward the sealing surface 332, e.g., to form the seal 334. Additionally, continued cooling of the first end 360a can recruit material from the second end 360b of the wax actuator 360 toward the first end 352a of the actuation assembly 340, e.g., causing the second end 360b to bend or “sink” as shown in FIG. 3A. In some embodiments, the bend/sink in the second end 360b of the wax actuator 360 deflects the second diaphragm 342b upwardly and/or toward the wax actuator 360, such as shown in FIG. 3A.

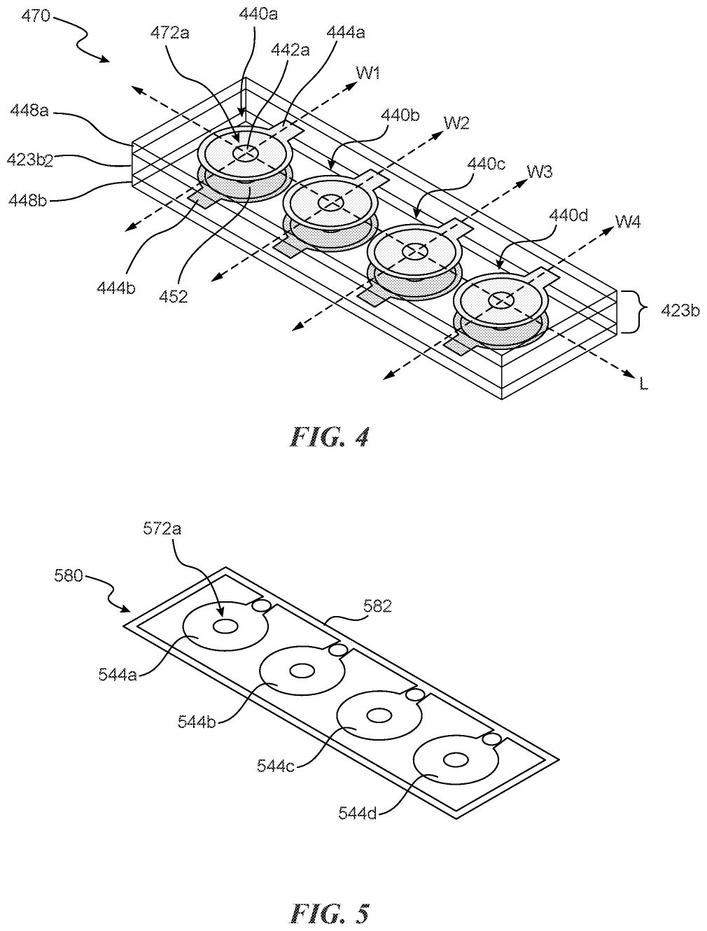

FIG. 4 illustrates an actuation assembly manifold 470 (“the manifold 470)”) including a plurality of actuation assemblies 440a-d and configured in accordance with embodiments of the present technology. Each of the actuation assemblies 440a-d can be generally similar to the actuation assembly 140 of FIGS. 1A-1D, the actuation assembly 240 of FIGS. 2A and 2B, and/or the actuation assembly 340 of FIGS. 3A and 3B, with like numbers (e.g., first actuation component 444a versus the first actuation component 144a of FIG. 1B) illustrating like elements. In the illustrated embodiment, the manifold 470 includes four actuation assemblies, e.g., a first actuation assembly 440a, a second actuation assembly 440b, a third actuation assembly 440c, and a fourth actuation assembly 440d. In other embodiments, the manifold 470 can include more or fewer actuation assemblies, such as at least one, two, three, five, or any other suitable number of actuation assemblies. In the illustrated embodiment, the actuation assemblies 440a-d are arranged in a single row. In other embodiments, the actuation assemblies 440a-d can be arranged in multiple rows, or in any other suitable arrangement.

The first actuation assembly 440a can include a body 452, a first actuation component 444a, a second actuation component 444b, and a first diaphragm 442a. The first actuation component 444a can include a first hole or aperture 472a configured (e.g., sized, positioned, and the like) such that the wax actuator (e.g., the wax actuator 360 of FIGS. 3A-3C) contained therein can interact with the first diaphragm 442a through the first hole 472a, e.g., to deform the first diaphragm 442a, as described previously with reference to at least FIGS. 3A-3C. Although not shown in the embodiments illustrated in FIGS. 1B-3C, in other embodiments, such as when the first actuation component 444a is formed from a relatively thin film or layer of material as shown in FIG. 4, the first hole 472a can allow at least a portion of the first actuation component 444a to bend or deform in concert with the first diaphragm 442a. Although not shown in FIG. 4, the first actuation assembly 440a can include a second diaphragm opposite the first diaphragm 442a, and the second actuation component 444b can include a second hole opposite the first hole 472a. Additionally, each of the second, third, and fourth actuation components 440b-d can be generally similar to or the same as the first actuation assembly 440a. For example, although not labeled in FIG. 4, the second, third, and fourth actuation components 440b-d can each respectively have at least a body generally similar to or the same as the body 452, a first actuation component generally similar to or the same as the first actuation component 444a, and a second actuation component generally similar to or the same as the second actuation component 444b.

In some embodiments, the manifold 470 can be a layer or plate within a flow control assembly, such as the flow control assembly 120 of FIGS. 1A-1D, the flow control assembly 220 of FIGS. 2A and 2B, or any other suitable flow control assembly. The manifold 470 can be at least a portion of an intermediate plate 423b of a flow control assembly, e.g., generally similar to the intermediate plate 223b of FIGS. 2A and 2B. In the illustrated embodiment, for example, the manifold 470 includes a first flexible layer 448a, a second flexible layer 448b, and an intermediate layer 423b₂ between the first and second flexible layers 448a-b. The actuation components 444a-b of each of the actuation assemblies 440a-d can be positioned on opposite sides of the intermediate layer 423b₂ and between the intermediate layer 423b₂ and one of the first flexible layer 448a or the second flexible layers 448b, e.g., the first actuation component 444a on a first side of the intermediate layer 423b₂ between the intermediate layer 423b₂ and the first flexible layer 448a and the second actuation component 444b on a second, opposite side of the intermediate layer 423b₂ between the intermediate layer 423b₂ and the second flexible layer 448b. In other embodiment, the manifold 470 can have other suitable configurations.

In operation, the actuation assembly manifold 470 can be used to control the flow of fluid in one or more directions. In some embodiments, the actuation assemblies 440a-d can control the flow of fluid in series, e.g., through a channel positioned in a first (e.g., lengthwise) direction L relative to the manifold 470. Additionally, or alternatively, individual ones of the actuation assemblies 440a-d can control the flow of fluid in parallel, e.g., through multiple channels positioned in respective second (e.g., widthwise) directions W1-4 relative to the manifold 470 (e.g., the first actuation assembly 440a in a first widthwise direction W1, the second actuation assembly 440b in a second widthwise direction W2, the third actuation assembly 440c in a third widthwise direction W3, and the fourth actuation assembly 440d in a fourth widthwise direction W4). In these and other embodiments, individual ones of the actuation assemblies 440a-d can be configured to control the flow of fluid in any other suitable direction relative to the manifold 470.

FIG. 5 is a perspective view of a plurality of actuation components 544a-d during a stage of a manufacturing process, in accordance with embodiments of the present technology. Each of the actuation components 544a-d can be generally similar to or the same as the actuation components 444a-d of FIG. 4, the actuation components 344a-d of FIGS. 3A and 3B, the actuation components 244a-b of FIGS. 2A and 2B, and/or the actuation components 144a-b of FIGS. 1A-1D, with like numbers (e.g., hole 572a versus the first hole 472a of FIG. 4) indicating like elements.

The actuation components 544a-d can be manufactured to form a single sheet 580 of material, such as gold, palladium, or any of the other suitable materials described herein. Although illustrated as including four actuation components 544a-d in FIG. 5, in other embodiments the single sheet 580 can include more or fewer actuation components 544a-d, such as at least one, two, three, five, or any other suitable number of actuation components. In some embodiments, individual ones of the actuation components 544a-d can be cut or etched from the single sheet 580, such as via laser etching or any other suitable cutting or material removal process known to those of skill in the art. In other embodiments, individual ones of the actuation components 544a-d can be formed using an additive manufacturing process, such as via chemical vapor deposition, or any other suitable additive manufacturing process known to those of skill in the art. In these and other embodiments, individual ones of the actuation components 544a-d can be coupled to a frame 582 during at least a portion of the manufacturing process. The frame 582 can be a same or different material than the actuation components 544a-d. In at least some embodiments, the single sheet 580 only includes the frame 582 and the actuation components 544a-d, such that there is no material (e.g., gaps or empty space) between the actuation components 544a-d. Individual ones of the actuation components 544a-d can be removed from the frame 582 before being added to an actuation assembly (e.g., the first actuation assembly 440a of FIG. 4, or any other actuation assembly described herein). In these and other embodiments, individual ones of the actuation components 544a-d can remain coupled to the frame 582 and the single sheet 580 can be used to form one or more actuation assemblies (e.g., the first, second, third, and/or fourth actuation assemblies 440a-d of FIG. 4). In such embodiments, for example, the single sheet 580 can be placed between the intermediate layer 423b₂ (FIG. 4) and the first flexible layer 448a (FIG. 4) as part of a process for manufacturing the actuation component assemblies 440a-d. Alternatively, or in addition, another single sheet 580 can be placed between the intermediate layer 423b₂ (FIG. 4) and the second flexible layer 448b (FIG. 4).

### EXAMPLES

Several aspects of the present technology are set forth in the following examples:
1. An actuation assembly for an adjustable shunting system for treating a patient, the actuation assembly comprising:
a diaphragm; and
a wax actuator operably coupled to the diaphragm and transitionable between a first configuration and a second configuration, wherein
   in the first configuration, the wax actuator has a first position configured to bias the diaphragm in a first direction; and
   in the second configuration, the wax actuator has a second position configured to bias the diaphragm in a second direction different than the first direction.
2. The actuation assembly of example 1 wherein the wax actuator includes a first end proximate the diaphragm and a second end opposite the first end, and wherein:

in the first configuration, the second end is curved inwardly toward the first end; and
in the second configuration, the first end of the wax actuator is curved inwardly toward the second end.
3. The actuation assembly of example 1 or example 2, further comprising:
a first actuation component thermally coupled to at least the first end of the wax actuator; and
a second actuation component thermally coupled to at least the second end of the wax actuator;
wherein
   the first actuation component is configured to receive first energy to transition the wax actuator from the first configuration toward the second configuration, and
   the second actuation component is configured to receive second energy to transition the wax actuator from the second configuration toward the first configuration.
4. The actuation assembly of example 3 wherein:
the first energy is first externally applied heat energy;
the second energy is second externally applied heat energy;
the first actuation component is configured to transfer the first heat energy to at least the first end of the wax actuator; and
the second actuation component is configured to transfer the second heat energy to at least the second end of the wax actuator.
5. The actuation assembly of example 4 wherein the first actuation component and the second actuation component are configured to transfer the first heat energy and the second heat energy, respectively, to the entire wax actuator to cause the entire wax actuator to transition from a solid material phase to a liquid material phase.
6. A flow control assembly for an adjustable shunting system for treating a patient, the flow control assembly comprising:
an actuation assembly including a wax actuator, wherein the wax actuator is configured to transition the actuation assembly between a first configuration and a second configuration;
a channel including a sealing surface; and
a diaphragm positioned at least partially between the actuation assembly and the sealing surface and operably coupled to the actuation assembly;
wherein
   when the actuation assembly is in the first configuration, the diaphragm forms a seal with the sealing surface; and
   when the actuation assembly is in the second configuration, the diaphragm is deflected away from the sealing surface.
7. The flow control assembly of example 6 wherein:
in the first configuration, the diaphragm at least partially prevents fluid flow through the channel; and
in the second configuration, the diaphragm permits fluid flow through the channel.
8. The flow control assembly of example 6 or example 7 wherein:
the channel includes a first channel portion and a second channel portion; and
the sealing surface is positioned between the first channel portion and the second channel portion;
in the first configuration, the diaphragm at least partially prevents fluid from flowing between the first channel portion and the second channel portion; and
in the second configuration, the diaphragm permits fluid to flow between the first channel portion and the second channel portion.
9. The flow control assembly of any of examples 6-8 wherein:
in the first configuration, the wax actuator applies a first force to the diaphragm in a first direction toward the sealing surface; and
in the second configuration, the wax actuator applies a second force to the diaphragm in a second direction away from the sealing surface.
10. The flow control assembly of example 9 wherein:
the wax actuator includes a first end proximate the diaphragm and a second end opposite the first end,
in the second configuration, the first end includes a first sink that deflects the first end toward the second end; and
in the first configuration, the second end includes a second sink that deflects the second end toward the first end.
11. The flow control assembly of example 10 wherein:
in the first configuration, the first force is based at least partially on the second sink; and
in the second configuration, the second force is based at least partially on the first sink.
12. The flow control assembly of any of examples 6-11 wherein the diaphragm is a first diaphragm and the channel is a first channel, and wherein the flow control assembly further comprises:
a second diaphragm opposite the first diaphragm and operably coupled to the actuation assembly; and
a second channel opposite the first channel and fluidly coupled to the second diaphragm.
13. The flow control assembly of example 12 wherein the first channel and the second channel are configured to receive fluid from a same body region.
14. The flow control assembly of example 12 or example 13 wherein the first channel is configured to allow fluid to flow fully through the flow control assembly and the second channel is configured to allow fluid to flow partially through the flow control assembly.
15. The flow control assembly of any of examples 12-14 wherein the second channel has a single opening.
16. An adjustable shunting system for treating a patient, the adjustable shunting system comprising:
a body having a first end portion and a second end portion;
a flow path through the body, wherein the flow path is defined at least partially by an inlet positioned proximate the first end portion and an outlet positioned proximate the second end portion; and
a flow control assembly fluidly coupled to the inlet and the outlet, wherein
   the flow control assembly includes a wax actuator operably coupled to a diaphragm, and
   the flow control assembly is transitionable between (i) a first configuration in which the wax actuator bends the diaphragm in a first direction to at least partially prevent fluid from flowing through a portion of the flow path, and (ii) a second configuration in which the wax actuator bends the diaphragm in a second direction opposite the first direction to allow fluid to flow through a portion of the flow path.
17. The adjustable shunting system of example 16 wherein:
in the first configuration, the wax actuator includes a first curvature in the first direction; and
in the second configuration, the wax actuator includes a second curvature in the second direction.
18. The adjustable shunting system of example 17 wherein:
the wax actuator includes a first end and a second end opposite the first end;
the first curvature includes a first concave curvature in the second end; and
the second curvature includes a first concave curvature in the first end.
19. The adjustable shunting system of any of examples 16-18 wherein:
the adjustable shunting system further includes a sealing surface at least partially align with the wax actuator;
the first direction is toward the sealing surface; and
the second direction is away from the sealing surface.
20. The adjustable shunting system of example 19 wherein, in the first configuration the diaphragm forms a seal with the sealing surface, and wherein, in the second configuration, the diaphragm is spaced apart from the sealing surface.
21. The adjustable shunting system of any of examples 16-20, further comprising one or more fluid resistance components.
22. The adjustable shunting system of example 21 wherein at least one of the one or more fluid resistance components are positioned between flow control assembly and the outlet.
23. A method for selectively controlling fluid flow through a shunting system, the method comprising:
applying energy to an actuation component of an actuation assembly of the shunting system;
transitioning a wax actuator of the actuation assembly from a first material phase to a second material phase; and
moving a diaphragm of the actuation assembly from (i) a first position in which the diaphragm at least partially blocks flow through a channel of the shunting system, to (ii) a second position in which the diaphragm permits flow through the channel.
24. The method of example 23 wherein moving the diaphragm from the first position to the second position includes forming a sink in a surface of the wax actuator.
25. The method of example 24 wherein transitioning the wax actuator from the first material phase to the second material phase includes causing the wax actuator to apply a force to the diaphragm, and wherein moving the diaphragm from the first position to the second position includes causing the diaphragm to move in response to the force.
26. The method of any of examples 23-25 wherein moving the diaphragm from the first position to the second position includes moving the diaphragm away from a sealing element of the flow control assembly.
27. The method of any of examples 23-26 wherein the actuation component is a first actuation component, the method further comprising:
applying energy to a second actuation component of the actuation assembly, and
moving the diaphragm from the second position toward the first position.
28. The method of example 27 wherein moving the diaphragm from the second position toward the first position includes forming a seal between the diaphragm and the sealing element.
29. The method of example 27 or example 28 wherein:
applying the energy to the first actuation component includes heating a first end of the wax actuator; and
applying the energy to the second actuation component includes heating a second end of the wax actuator, the second end opposite the first end.
30. The method of any of examples 23-29, further comprising allowing the wax actuator to transition from the second material phase to the first material phase.
31. The method of any of examples 23-30 wherein, the wax actuator is a solid in the first material phase, the wax actuator is a liquid in the second material phase, and transitioning the wax actuator from the first material phase to the second material phase includes heating the wax actuator above a solid-liquid transition temperature.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An actuation assembly for an adjustable shunting system for treating a patient, the actuation assembly comprising:
a diaphragm; and
a wax actuator operably coupled to the diaphragm and transitionable between a first configuration and a second configuration, wherein
in the first configuration, the wax actuator has a first position configured to bias the diaphragm in a first direction; and
in the second configuration, the wax actuator has a second position configured to bias the diaphragm in a second direction different than the first direction.

2. The actuation assembly of claim 1 wherein the wax actuator includes a first end proximate the diaphragm and a second end opposite the first end, and wherein:
in the first configuration, the second end is curved inwardly toward the first end; and
in the second configuration, the first end of the wax actuator is curved inwardly toward the second end.

3. The actuation assembly of claim 1, further comprising:
a first actuation component thermally coupled to at least the first end of the wax actuator; and
a second actuation component thermally coupled to at least the second end of the wax actuator;
wherein
the first actuation component is configured to receive first energy to transition the wax actuator from the first configuration toward the second configuration, and
the second actuation component is configured to receive second energy to transition the wax actuator from the second configuration toward the first configuration.

4. The actuation assembly of claim 3 wherein:
the first energy is first externally applied heat energy;
the second energy is second externally applied heat energy;
the first actuation component is configured to transfer the first heat energy to at least the first end of the wax actuator; and
the second actuation component is configured to transfer the second heat energy to at least the second end of the wax actuator.

5. The actuation assembly of claim 4 wherein the first actuation component and the second actuation component are configured to transfer the first heat energy and the second heat energy, respectively, to the entire wax actuator to cause the entire wax actuator to transition from a solid material phase to a liquid material phase.

6. A flow control assembly for an adjustable shunting system for treating a patient, the flow control assembly comprising:
an actuation assembly including a wax actuator, wherein the wax actuator is configured to transition the actuation assembly between a first configuration and a second configuration;
a channel including a sealing surface; and
a diaphragm positioned at least partially between the actuation assembly and the sealing surface and operably coupled to the actuation assembly;
wherein
when the actuation assembly is in the first configuration, the diaphragm forms a seal with the sealing surface; and
when the actuation assembly is in the second configuration, the diaphragm is deflected away from the sealing surface.

7. The flow control assembly of claim 6 wherein:
in the first configuration, the diaphragm at least partially prevents fluid flow through the channel; and
in the second configuration, the diaphragm permits fluid flow through the channel.

8. The flow control assembly of claim 6 wherein:
the channel includes a first channel portion and a second channel portion; and
the sealing surface is positioned between the first channel portion and the second channel portion;
in the first configuration, the diaphragm at least partially prevents fluid from flowing between the first channel portion and the second channel portion; and
in the second configuration, the diaphragm permits fluid to flow between the first channel portion and the second channel portion.

9. The flow control assembly of claim 6 wherein:
in the first configuration, the wax actuator applies a first force to the diaphragm in a first direction toward the sealing surface; and
in the second configuration, the wax actuator applies a second force to the diaphragm in a second direction away from the sealing surface.

10. The flow control assembly of claim 9 wherein:
the wax actuator includes a first end proximate the diaphragm and a second end opposite the first end,
in the second configuration, the first end includes a first sink that deflects the first end toward the second end; and
in the first configuration, the second end includes a second sink that deflects the second end toward the first end.

11. The flow control assembly of claim 10 wherein:
in the first configuration, the first force is based at least partially on the second sink; and
in the second configuration, the second force is based at least partially on the first sink.

12. The flow control assembly of claim 6 wherein the diaphragm is a first diaphragm and the channel is a first channel, and wherein the flow control assembly further comprises:
a second diaphragm opposite the first diaphragm and operably coupled to the actuation assembly; and
a second channel opposite the first channel and fluidly coupled to the second diaphragm.

13. The flow control assembly of claim 12 wherein the first channel and the second channel are configured to receive fluid from a same body region.

14. The flow control assembly of claim 12 wherein the first channel is configured to allow fluid to flow fully through the flow control assembly and the second channel is configured to allow fluid to flow partially through the flow control assembly.

15. The flow control assembly of claim 12 wherein the second channel has a single opening.

16. An adjustable shunting system for treating a patient, the adjustable shunting system comprising:
a body having a first end portion and a second end portion;
a flow path through the body, wherein the flow path is defined at least partially by an inlet positioned proximate the first end portion and an outlet positioned proximate the second end portion; and
a flow control assembly fluidly coupled to the inlet and the outlet, wherein
the flow control assembly includes a wax actuator operably coupled to a diaphragm, and
the flow control assembly is transitionable between (i) a first configuration in which the wax actuator bends the diaphragm in a first direction to at least partially prevent fluid from flowing through a portion of the flow path, and (ii) a second configuration in which the wax actuator bends the diaphragm in a second direction opposite the first direction to allow fluid to flow through a portion of the flow path.

17. The adjustable shunting system of claim 16 wherein:
in the first configuration, the wax actuator includes a first curvature in the first direction; and
in the second configuration, the wax actuator includes a second curvature in the second direction.

18. The adjustable shunting system of claim 17 wherein:
the wax actuator includes a first end and a second end opposite the first end;
the first curvature includes a first concave curvature in the second end; and
the second curvature includes a first concave curvature in the first end.

19. The adjustable shunting system of claim 16 wherein:
the adjustable shunting system further includes a sealing surface at least partially align with the wax actuator;
the first direction is toward the sealing surface; and
the second direction is away from the sealing surface.

20. The adjustable shunting system of claim 19 wherein, in the first configuration the diaphragm forms a seal with the sealing surface, and wherein, in the second configuration, the diaphragm is spaced apart from the sealing surface.

21. The adjustable shunting system of claim 16, further comprising one or more fluid resistance components.

22. The adjustable shunting system of claim 21 wherein at least one of the one or more fluid resistance components are positioned between flow control assembly and the outlet.

23. A method for selectively controlling fluid flow through a shunting system, the method comprising:
applying energy to an actuation component of an actuation assembly of the shunting system;
transitioning a wax actuator of the actuation assembly from a first material phase to a second material phase; and
moving a diaphragm of the actuation assembly from (i) a first position in which the diaphragm at least partially blocks flow through a channel of the shunting system, to (ii) a second position in which the diaphragm permits flow through the channel.

24. The method of claim 23 wherein moving the diaphragm from the first position to the second position includes forming a sink in a surface of the wax actuator.

25. The method of claim 24 wherein transitioning the wax actuator from the first material phase to the second material phase includes causing the wax actuator to apply a force to the diaphragm, and wherein moving the diaphragm from the first position to the second position includes causing the diaphragm to move in response to the force.

26. The method of claim 23 wherein moving the diaphragm from the first position to the second position includes moving the diaphragm away from a sealing element of the flow control assembly.

27. The method of claim 23 wherein the actuation component is a first actuation component, the method further comprising:
applying energy to a second actuation component of the actuation assembly, and
moving the diaphragm from the second position toward the first position.

28. The method of claim 27 wherein moving the diaphragm from the second position toward the first position includes forming a seal between the diaphragm and the sealing element.

29. The method of claim 27 wherein:
applying the energy to the first actuation component includes heating a first end of the wax actuator; and
applying the energy to the second actuation component includes heating a second end of the wax actuator, the second end opposite the first end.

30. The method of claim 23, further comprising allowing the wax actuator to transition from the second material phase to the first material phase.

31. The method of claim 23 wherein, the wax actuator is a solid in the first material phase, the wax actuator is a liquid in the second material phase, and transitioning the wax actuator from the first material phase to the second material phase includes heating the wax actuator above a solid-liquid transition temperature.
